(19) United States
(12) Patent Application Publication
(10) Pub. No.: US 2010/0056961 A1
(43) Pub. Date: Mar. 4, 2010

(54) METHOD AND DEVICE FOR DETECTING DEGLUTITION IN BABIES

(76) Inventor: Luis Maria Voloschin, Buenos Aires (AR)

Correspondence Address: YOUNG & THOMPSON, 209 Madison Street, Suite 500, Alexandria, VA, 22314, US

(21) Appl. No.: 12/513,114

(22) PCT Filed: Oct. 25, 2007
(86) PCT No.: PCT/IB2007/054349

§ 371 Date: May 26, 2009

(30) Foreign Application Priority Data
Oct. 30, 2006 (AR) P20060104751

(51) Int. Cl. A61B 5/11

(52) U.S. Cl. 600/593

(57) ABSTRACT

A method for detecting deglutition includes steps of: generating in the computer memory a number of addresses perfectly identifiable from a start point on which electrical transients received from the sensor element will be recorded; shooting the first computer memory scan upon receiving the signal of the first suction movement detected by the cutaneous electrodes or pressure transducers; recording electrical transients of records from the microphone or movement sensors at the different memory addresses: consider as a fresh start for recording electrical transients from the microphone every new signal of a new suction movement; averaging the electrical transients of records from the sensor element at different memory addresses; compare averaged electrical transients during apneas to normal breathing (to show that the ingested milk was swallowed during the times when the infant develops apneas). The device includes: a respiration sensor element; a suction sensor element; and a deglutition sensor element.

### FIELD OF THE INVENTION

The present invention relates to a method for detecting deglutition in infants and to a suction, deglutition and respiration coordination diagnosis and recording device during milk ingestion of preterm infants using the same. The method allows diagnosing and recording when the preterm infant is capable of coordinating respiration and deglutition, providing a document of the measurements recorded, such as in case of an electrocardiogram or electroencephalogram, with a certain and incontrovertible legal value.

The method of this invention allows generating a document that diagnosis in a certain and incontrovertible manner and with legal value that the infant is capable of coordinating respiration and deglutition.

### BACKGROUND ART

Air going to the lungs as food going to the stomach are conducted through the pharynx, and so it is necessary coordination between respiration and deglutition in a way food can reach the stomach when degluted and air to the lungs when breathed. The reflexes allowing coordination between respiration and deglutition are already mature and work correctly at the time of birth. However, when babies are born prematurely the coordination reflexes between respiration and deglutition are not mature yet and it is necessary to feed them artificially so that they do not run the risk of breathing at the time they swallow due to lack of coordination and so ingested milk goes to the lungs instead of the stomach.

One of the users of this instrument are maternities having neonatology services with intensive care units for preterm babies, as use of the same will allow a reduction in admission costs. It is important to note that neonatology services do not discharge a preterm infant until the capability of coordinating respiration-deglutition has been developed. In case after being discharged a preterm infant suffers an accident during feeding and that causes serious consequences, his parents may take legal action on the grounds of medical malpractice against the medical institution for having discharged a patient not able to coordinate respiration-deglutition yet. To avoid medical malpractice actions maternities assume higher costs and delay discharges to allow the infant to mature in order to ensure and guarantee that the preterm infant has correct respiration-deglutition coordination.

These delays to discharge the infant from hospital, that increase the admission time in intensive care units, are due to the fact that at present Neonatology Services do not have a legal evidence that shows a judge in a certain and incontrovertible manner that the preterm infant already coordinated respiration-deglutition at the time of discharge avoiding in this way medical malpractice actions. Therefore, the use of the present device will allow Maternities to avoid additional days of admission to intensive care units and reduce considerably the treatment costs of preterm babies.

Other users of the present device are Pediatric Services and pediatric physicians willing to diagnose and properly document if an infant coordinates adequately respiration-deglutition during milk ingestion.

In relation with the previous art, the application of some thermistors, electrodes and elements that are introduced into the body of the infant to diagnose if he is capable of swallowing his own ingestion are known. Other devices avoid introduction of elements within the infant replacing the same with microphones that try to sense the sounds typical of deglutition. One of the big drawbacks found are the presence of sounds strange to deglutition that prevent from ensuring with no doubts that said ingestion was taking place. By surface sensors, it is possible to detect trachea, glottis or hyoid bone movements generated at the time of deglutition. There are also other movements strange to deglutition that prevent from establishing that deglutition is taking place.

Diagnosis and Recording Device

The proposed device of the present invention uses a non-invasive method for the infant and records:
1.1. Respiration preferably by a thermistor located in the infant naris;
1.2. Suction oral motor activity preferably by two surface cutaneous electrodes that rest on the skin, one on the cheek and the other under the chin and in case of using a feeding bottle by an elastic tube inside the teat.
1.3. Deglutition preferably by a flat mini-microphone attached to the skin in the cervical region lateral to the larynx that allows detecting the sound produced when swallowing the food bolus. In addition, other sensors can be used to detect hyoid bone, glottis or trachea movements generated during deglutition, etc. The data obtained is digitalized and later used in Time-Locked Signal Automatic Processing Programs.

Recording of Respiration

As regards respiration and the record forms found in the same, the following classes can be defined:
1.1. Normal respiration: the line is regular and a short delay between inhalation and exhalation is observed.
1.2. Respiration with apnea: to coordinate respiration with deglutition the infant has to ‘stop’ breathing at the time of swallowing. That is to say the infant obstructs respiration (Apnea) at the time of deglutition,

When the infant ‘obstructs’ respiration, he increases the delay between Inhalation and Exhalation. The duration of the delay varies according to the number of deglutitions the infant has to ‘hold’ his breath. Each deglutition takes about 1 second, so if the infant holds his breath only one deglutition the delay between inhalation and exhalation will take 1 second; moreover, if the infant holds his breath multiple deglutitions, for example 5 deglutitions, the delay between inhalation and exhalation will take about 5 seconds. Generally, an infant ‘holds’ several deglutitions without swallowing and because of that the respiration record line is irregular with delays of different duration (short, intermediate and long, etc.). The ‘irregular’ respiration record line observed when the infant breathes doing Apneas is very apparent and ease to diagnose. Additionally, the simultaneous recording of the suction oral motor activity shows that at that moment the infant is performing a ‘nutritive’ oral motor activity that, as hereinafter mentioned, is accompanied by deglutitions time-locked to said ‘nutritive’ oral motor activity.

Recording of Oral Motor Activity During Suction

In relation with recording suction oral motor activity, it is possible to perform the test during either breast-feeding or bottle-feeding. In case of performing the test during breast feeding, said electrodes can be used, and in case of performing the test during bottle feeding, methods capable of detecting pressure changes caused by the infant during suction can be used, for example an elastic tube full of water with one of its ends within the teat and the other end connected to a pressure transducer.

When the infant is breast or bottle fed, he starts two different oral motor activities that are very characteristic:
1.1. Nutritive Oral Motor Activity: This suction activity is performed at the time the infant is ingesting milk. It comprises a series of rhythmical oral suction movements with a frequency of about 1 Hz that are executed ‘continuously’. During this type of suction activity, many deglutitions are produced simultaneously, also in a continued manner, so that each of the suction movements is ‘time-locked’ with each deglutition.
1.2. Non-nutritive Oral Motor Activity: This suction activity is performed when the infant is not ingesting milk; it is considered a ‘calm down action’. It comprises a series of rhythmical oral suction movements with a frequency of about 2 Hz, and executed for the most part ‘intermittently’, that is to say with short pauses between activity trains. During this type of suction activity, only very few ‘isolated’ deglutitions are observed that are not time-locked to the suction movements.

The Suction Motor Oral Activity Recording Systems either Cutaneous Surface Electrodes or Pressure Transducer allow to clearly identifying these two activities executed by the infant when he is breast or bottle-fed. In the case of a feeding bottle, these two activities are observed in a more schematic manner as in a feeding bottle milk is always available while in the breast the access the availability of milk is variable.

With reference to the different recording systems that allow detecting and diagnosing said ‘Nutritive’ or ‘Non-Nutritive’ suction oral motor activities, we can express as follows:

1. By cutaneous Surface electrodes (Orokinetogram; Voloschin and others): A non-invasive method detects the oral movements made by the infant during suction, without interposing any element between the mother's nipple and the infant's tongue. The electrodes are supported: one on the skin of the face in the area of the cheek and the other on the skin of the face in the area under the tongue. When the infant suckles, ‘very little’ electric variations between both cutaneous electrodes are produced. The electric variations are detected and recorded by the device. The continuous recording of the electric variations shows the rhythmic activity performed by the infant during suction (‘low frequency or nutritive oral movements’ or ‘higher frequency or non-nutritive oral movements’). This method is very suitable for analyzing the milk ingestion during ‘natural breast feeding’ as it does not cause any interference inside the infant's mouth.

2. By a pressure detection device (Conventional method mostly used at present): It is also a non invasive method, that detects pressure changes caused by the infant during suction, but this method interposes an elastic thin tube full of water between the mother's nipple and the infant's tongue. One of the ends of the tube is connected to a pressure transducer that detects and measures pressure changes while the other end remains inside the infant's mouth. In the case of breast-feeding, the tube rests on the nipple and in the case of bottle-feeding, the tube us located inside the teat. The use of a feeding bottle has several advantages: a) having free access to the milk, the infant performs nutritive and non-nutritive rhythmic activities in a more schematic manner during breast-feeding; b) being the feeding bottle a separate object, it is possible to design feeding bottles having incorporated sensors or sophisticated instruments.

When the infant suckles, positive and negative pressure variations are produced. The pressure variations are detected by the pressure transducer, through the elastic tube interposed between the nipple and the infant's tongue or inside the bottle's teat. The continuous recording of pressure variations shows the rhythmic activity performed by the infant during suction (‘nutritive rhythm’ or ‘non-nutritive rhythm’).

The use of this method allows developing many beneficial sophisticated constructive technologies that certainly we are using in producing our device, especially if the milk ingestion analysis is performed during bottle feeding as in this case the elastic tube is located inside the teat and the infant does not feel it at all.

The elements used in these three measurements, i.e., preferably, thermistor, cutaneous electrodes, teats with feeding bottles and flat microphone, are presently commercialized and may be obtained from measuring or medical instrument shops.

The method for recording suction by cutaneous electrodes on the cheek and under the chin has resulted from a design owned by us, known as orokinetogram. The same has been published in the Journal of reproduction and fertility 114, 210-224. 1998 (Voloschin et al. ‘A new tool for measuring the suckling stimulus during breastfeeding in humans: the orokinetogram and the fourrier series.’).

The records of respiratory movements and suction oral movements are very clear and from said records, it is possible to identify easily inhalation, exhalation, and nutritive and non-nutritive suction rhythms.

Method for Detecting Deglutition in an Infant

Deglutition may be detected by the sound it produces or by the movement of the trachea, glottis, or bones, like the hyoid bone, that displace during deglutition. It is convenient to detect the sound of deglutition, as a wide range of suitable microphones is commercially available. In this description, we show how to detect deglutition by the sound it produces.

The records of the different sounds detected with the microphone resting on the cervical region correspond on one hand to the sound produced when swallowing the food bolus but at the same time other sounds produced simultaneously with deglutition by respiration and muscular movements are detected and recorded. According to the intensity, the sounds adjacent to deglutition may hide the sounds produced by deglutition and in this manner; the sound of deglutition recorded by the microphone is confused with the record of adjacent sounds preventing identification of the same.

### DETAILED DESCRIPTION OF THE INVENTION

In order to detect deglutition by identifying the sound record that specifically corresponds to deglutition, Time Locked Signal Processing Technologies such as signal averaging computer technologies (superposing or adding responses), described in this patent application, or other technologies as adaptative filters are needed, considering that they allow to remove sound records coming from other non-specific adjacent sources when swallowing such as those produced by respiration or other random sounds.

In the non-weaned infant milk deglutition is a direct consequence of the ‘nutritive’ oral motor activity performed by the infant during nipple suckling and both occur sequentially and with a constant delay between the suction movement and the time of deglutition. That is to say, ‘nutritive’ oral movements and deglutitions are ‘time-locked’.

It is important to note that there are important works such as the one published in Development Medicine & Child Neurology 1997, 39. 534-542 entitled ‘Deglutition apnea as indicator of maturation of suckle feeding in bottle-fed preterm infants.’ by the investigators Hanlon M B, Tripp J H, Ellis R E, Flack F C, Selley W G, Shoesmith H J, that allow to confirm the ‘time-locking’ between suction and start of deglutition.

Method for Detecting Deglutition Sound by Computer Time-Locked Signal Averaging Technologies

To process averaging, two signals are used:

a) The ‘reference’ signal is the beginning of a suction movement. By using voltage level variation detectors, an electric pulse is ‘shot’ at the time an electrical transient corresponding to a suction movement starts. This electric pulse is also used as a ‘shot’ to scan the computer memory. As previously discussed, the suction movement is recorded by means of two cutaneous electrodes resting on the skin, one on the cheek and the other under de chin (in case of using a feeding bottle, the suction movement is recorded by a tube full of water located inside the bottle's teat and connected to a pressure transducer).

b) The ‘specific’ signal is the sound of deglutition that appears sequentially to the suction movement. The electrical transients of the microphone produced by the sound of deglutition are recorded in memory. Said electrical transients remain recorded in the same computer memory addresses for being ‘time-locked’ with the scanning start. The signal averaging (superposing or adding responses) is a useful technology to detect specific responses with respect to a noise background that has the recording system and which is Gaussian or randomly distributed and hides the sound produced by deglutition preventing an accurate identification thereof.

The term ‘noise’ herein means sound records that are not related to the sequence first ‘nutritive’ suction movement—first sound of deglutition; second ‘nutritive’ suction movement—second sound of deglutition . . . ‘N’ ‘nutritive’ suction movement—‘N’ sound of deglutition.

To specifically identify the sound of deglutition and remove random sounds, electrical transients of the records coming from the microphone shall be averaged. To that end, a software program is used that shoots the first computer memory scan when the first ‘nutritive’ suction movement starts.

Once the first scanning starts, the electrical transients of the sounds coming from the microphone are successively recorded at the different addresses of the memory. In this way, in this first scanning, a copy of all the signals obtained by the microphone is recorded.

The second ‘nutritive’ suction movement shoots the start of the second scanning and this time the electrical transients of the sounds corresponding to the second deglutition are averaged at the different memory addresses with the records of the previous scanning. Thus, the record stored in the computer memory after the second scanning corresponds to the average of electrical transients of the sounds stored during the first and second scanning.

The electrical transients of the microphone corresponding to the sound of deglutition are always recorded at the same computer memory addresses for being time-locked to the start of the scan shot by the start of the ‘nutritive’ suction movement and consequently their average value progressively increases as successive suctions occur, while the random or Gaussian electrical transients which are not time-locked to the start of suction will not always fall at the same computer addresses and consequently their average value decreases as scanning occurs.

Therefore, as successive deglutitions are averaged, the average value of the electrical transients corresponding to deglutition will increase, and so they can be clearly distinguished from the other electrical transients not related to deglutition (noises). In this manner, it is possible to identify deglutition electrical transients in a certain and incontrovertible manner.

If instead of detecting the sound of deglutition, it is desired to detect the movements of the trachea, glottis, or hyoid bone produced during deglutition, in this case it is used as ‘specific signal’ the electrical transients from the movement sensor. Instead of averaging the electrical transients from the sound captured by the microphone, electrical transients from the sensor that detects the movements of the trachea, glottis, hyoid bone or any other tegument which movement accompanies deglutition are averaged. In this way, as with the sound, the use of computer time-locked signal averaging technologies allows eliminating electrical transients from random movements that are not related to deglutition and so electrical transients corresponding to the movements produced by deglutition are distinguished and identified in a clear and incontrovertible manner. As previously discussed, to detect deglutition, preferably its sound is captured by microphones as it is possible to benefit from the wide range of new technologies in the manufacturing of microphones presently available in the market.

Deglutition as well as respiration and suction oral activity recordings allow diagnosing coordination between respiration and deglutition in infants.

Diagnosing and Recording Device

The device of the present invention, in a preferred form of operation, digitalizes signals and obtains and stores the same in memory at 15 seconds intervals. That is to say, recording data can be analyzed at 15 seconds intervals (4 intervals per minute). Consequently, if the infant has been suckling for 10 minutes there are 40 fractions recorded that can be separately and successively viewed. The records of respiratory movements, suction oral movements and larynx movements captured by the microphone are simultaneously recorded on each of the 15-second intervals.

Each of the 15-second intervals can be copied and the copy is separately stored. The device comprises two different locations to store the copies. At one location, the system operator stores copies of all those 15-second intervals wherein he diagnosed that the infant is breathing ‘normally’ creating in this way a ‘Group of Records with Normal Respirations’. At the second location, the system operator stores copies of all those 15-second intervals wherein he diagnosed that the infant developed apneas forming in this way a ‘Group of Records with Apnea Respiration’. The device processes separately the records stored at each of these two groups. The separate processing of the data from the ‘Group of Records with Normal Respirations’ and the ‘Group of Records with Apnea Respiration’ allows comparing the results obtained in both groups. This way of grouping and analyzing allows to evidence that the method for detecting deglutition according to the invention detects the sound of deglutition only at the ‘Group of Records with Apnea Respiration’ that is to say when the infant develops apneas. This shows that the infant, that we are monitoring, only swallows when he simultaneously ‘holds’ his breath (apnea). Therefore, it shows that this infant is already coordinating respiration with deglutition as he stops breathing (apnea) when he swallows so that milk does not end in the lungs.

This device allows generating a document to diagnose that the infant is already coordinating respiration and deglutition. Creation of the document comprises the following steps:

First Step

Continuous and simultaneous acquiring of records of:
0 1.1. Respiration:
1.2. suction and
1.3. The sound from the microphone.

The infant is monitored while breast of bottle-fed. Records are digitally recorded.

Second Step

Classification of recorded records.

The system operator must, manually, identify, classify, store and save separately the recorded records in two groups or sets of records, as follows:
1.a. ‘Group of Records with Normal Respirations’
1.b. ‘Group of Records with Apnea Respiration’

Third Step

Separate analysis of the ‘Groups of Records’.

a. Analysis of the ‘Group of Records with Normal Respirations’

In normal babies, the following results will be observed.
1.a. Record of Respiratory Movements

A Regular Respiratory Rhythm Line with very short delay between inhalation and exhalation is recorded and diagnosed.
1.b. Record of Suction Oral Movements

A ‘Non-Nutritive’ Suction Oral Motor Activity or Pauses of the suction activity is recorded and diagnosed.
1.c. Record of Sounds captured by the Microphone

The method for detecting deglutition used in the present invention DOES NOT detect the sound of deglutition in this recorded group of records.

B. Analysis of the ‘Group of Records with Apnea Respiration’

In normal babies, the following results will be observed.
1.a. Record of Respiratory Movements

It is recorded and diagnosed an Irregular Respiratory Rhythm Line with delays between inhalation and exhalation of different duration (short, intermediate, long, etc.), that are time related to the simultaneous record of ‘Nutritive’ Suction Oral Movements.
1.b. Record of Suction Oral Movements

A ‘Nutritive’ Suction Oral Motor Activity is recorded and diagnosed.
1.c. Record of Sounds captured by the Microphone

The method for detecting deglutition used in the present invention DOES detect the sound of deglutition in this recorded group of records.

Fourth Step:

Making of the document that shows the infant is capable of coordinating respiration with deglutition.

Said document comprises:
1.a) Examples of simultaneous Records of Respiration and Suction obtained in each of the two Groups.
1.b) The results obtained for each of the two Groups, with the method for detecting deglutition of the invention.
1.c) Amount of milk ingested, which is calculated be the infant weight difference before and after being breast or bottler fed or by measuring the centimeters of milk consumed from a feeding bottle.
1.d) Diagnosis of the Study (example of ‘text’ in normal infants):

‘The respiration, suction and deglutition simultaneous records showed that deglutition was only detected when the infant has respiratory apneas that are time related with nutritive suctions. Therefore, the infant under study is capable of Coordinating Suction, Deglutition, and Respiration during Milk Ingestion’.

The method for determining sound or movements produced by an infant deglutition comprises the following steps:
1.a. Generating in the computer memory where signals, preferably sounds, will be recorded, a number of addresses or parts perfectly identifiable from a start point on which electrical transients received from the microphone will be recorded;
1.b. Shooting the first computer memory scan upon receiving the signal of the first suction movement detected by the cutaneous electrodes in case of breast feeding or pressure transducers in case of using a feeding bottle.
1.c. Recording the electrical transients of the records from the microphone or the movement sensors at the different memory addresses:
1.d. Consider as a fresh start for recording electrical transients from the microphone or movement sensors every new signal of a new suction movement;
1.e. Averaging the electrical transients of the records from the microphone or movement sensors at the different memory addresses.
1.f. Compare the results of the averaged electrical transients during the periods of time when the infant breathes developing apneas to those obtained when the infant breathes normally (to show that the ingested milk was swallowed during the times when the infant develops apneas).

Said sensor means are preferably a microphone or movement sensor and cutaneous electrodes or pressure transducers.

The device for diagnosing and recording coordination of suction, deglutition and respiration that applies the method of claim 1 is comprised by: a) at least a respiration sensor means; b) at least a suction sensor means and; c) at least a deglutition sensor means.

Preferably but not exclusively, said respiration sensor means is a thermistor located at the infant's naris to detect rhythmic chances produced in inhalation and exhalation; the suction sensor means is made of two cutaneous electrodes resting on the skin, one on the cheek and the other under the chin, or a pressure transducer in case of using a feeding bottle; and the deglutition sensor means is a flat mini-microphone.

It is apparent that in the practice of the invention, modifications to certain construction and design details can be made, as well as using different time-locked signal processing systems, for example, the use of adaptative filters, etc., to detect deglutition without departing from the basic principles of the invention as clearly expressed in the following claims.

1-8. (canceled)

9. A method for detecting the deglutition signal of an infant wherein time-locked signal averaging technologies are used by employing a computer to record the electrical transients from sensor means, characterized in that comprises the following steps:
a. Generating in a computer memory where signals, preferably sounds, will be recorded, a number of addresses or parts perfectly identifiable from a start point on which electrical transients received from the sensor means will be recorded;
b. Triggering the first computer memory scan upon receiving the signal of the first suction movement detected by the cutaneous electrodes or pressure transducers;
c. Recording the electrical transients of the records from the sensor means or the movement sensors at the different memory addresses:
d. Retriggering the recording electrical transients from the sensor means every new signal of a new suction movement;
e. Averaging the electrical transients of the records from the sensor means at the different memory addresses.
f. Comparing the results of the averaged electrical transients to the periods of time when the infant breathes developing apneas.

10. A method for detecting the deglutition signal in an infant according to claim 9, characterized in that said sensor means is a microphone.

11. A method for detecting the deglutition signal in an infant according to claim 9, characterized in that said sensor means are movement sensors in case of detecting trachea, glottis or bone-muscle movements and cutaneous electrodes or pressure transducers.

12. A suction, deglutition and respiration coordination diagnosis and recording device for carrying out the method of claim 9, characterized by comprising:
a. A computer, a computer memory, and wherein the sensor means comprised of at least one respiration sensor;
b. At least one suction sensor; and
c. At least one deglutition sensor.

13. A device as claimed in claim 9, wherein said respiration sensor is a thermistor located at the infant's naris.

14. A device as claimed in claim 9, wherein said suction sensor is comprised by two cutaneous electrodes resting on the skin, one on the cheek and the other under the chin, or an elastic tube located inside the feeding bottle's teat and connected to a pressure transducer.

15. A device as claimed in claim 9, wherein said deglutition sensor means is a flat mini-microphone.

16. A device as claimed in claim 9, wherein said deglutition sensor means is a trachea, glottis, bone-muscle movement detector.
